(19) United States
(12) Patent Application Publication
(10) Pub. No.: US 2025/0210148 A1
(43) Pub. Date: Jun. 26, 2025

(54) METHOD OF PRODUCING DATA-ENCODED NUCLEIC ACID AND NUCLEIC ACID PRODUCED BY THE SAME

(71) Applicant: Samsung Electronics Co., Ltd., Suwon-si (KR)

(72) Inventors: Nagjong Kim, Suwon-si (KR); Sunghaeng Lee, Suwon-si (KR); Younsuk Choi, Suwon-si (KR)

(21) Appl. No.: 18/746,507

(22) Filed: Jun. 18, 2024

(30) Foreign Application Priority Data
Dec. 21, 2023 (KR) 10-2023-0188510

(51) Int. Cl. G16B 50/30; C12N 15/10

(52) U.S. Cl. CPC G16B 50/30; C12N 15/1068; C12N 15/1089

(57) ABSTRACT

Provided are a method for producing a data-encoded nucleic acid by using a template-dependent nucleic acid polymerase, and a data-encoded nucleic acid produced thereby.

### CROSS-REFERENCE TO RELATED APPLICATION

This application is based on and claims priority under 35 U.S.C. § 119, and all of the benefits accruing therefrom, to Korean Patent Application No. 10-2023-0188510, filed on Dec. 21, 2023, in the Korean Intellectual Property Office, the content of which is incorporated by reference herein in its entirety.

### SEQUENCE LISTING

The Instant Application contains a Sequence Listing which has been submitted electronically in XML format and is hereby incorporated by reference in its entirety. Said XML copy, created on Mar. 20, 2024, is named “7.SI74494US_Sequence List” and is 2,751 bytes in size. The Sequence Listing does not go beyond the disclosure in the application as filed.

### BACKGROUND

### 1. Field

The present disclosure relates to a method of producing a data-encoded nucleic acid, and a data-encoded nucleic acid produced by the same.

### 2. Description of the Related Art

Current electronic information storage technologies are based on memory chips made from silicon. However, silicon-based technologies have several drawbacks: they are estimated to have a scaling limit of about 10 nm, and, since silicon is not a renewable resource, the resources for these technologies may be depleted.

Therefore, there is a need for data storage technologies that enable storage of information in a more compact form, are renewable, and are available in large quantities. Nucleic acids have drawn attention as data storage substances both because they are a renewable resource and due to their ability to encode information in a small space. However, there is still a need to develop a method of effectively producing data-encoded nucleic acids.

### SUMMARY

Provided herein is a method for producing a data-encoded nucleic acid, the method including performing one or more repeating cycle, each cycle comprising (i) an extension reaction and (ii) a removing monomer process, wherein the extension reaction (i) is a polymerase extension of a data-encoded template DNA, wherein the polymerase is incubated with a reaction mixture comprising a data-encoded template, a buffer, a primer having a region complementary to the template, and a first nucleotide monomer to produce an incubated reaction mixture, wherein the removing monomer process (ii) removes unreacted first nucleotide monomer from the incubated reaction mixture in (i) to produce a removed reaction mixture, and mixing a second nucleotide monomer to prepare a second extension reaction mixture, wherein the template includes a coding region with a sequence complementary to the data-encoded nucleic acid and a primer-binding region complementary to a sequence of the primer. In an aspect the data-encoded nucleic acid includes an aminoallyl nucleotide. In another aspect, the reaction mixture includes a modified nucleotide after performing the repeating cycle once or more.

Provided is a data-encoded nucleic acid produced by the method.

Additional aspects will be set forth in part in the description which follows and, in part, will be apparent from the description, or may be learned by practice of the presented embodiments of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIGS. 1A and 1B show results of PAGE (upper) analysis and MALDI-TOF (lower) analysis of a product obtained by performing one extension reaction of a reaction mixture including a forward primer, a Bst polymerase, and a monomer;

FIGS. 1C and 1D show results of PAGE-gel analysis and MALDI-TOF analysis of a product obtained by further performing one extension reaction of the reaction mixture including washed reaction products of FIG. 1A or 1B, the Bst polymerase, and a monomer;

FIG. 2 shows results of PAGE analysis of a product obtained by performing five extension reactions of a reaction mixture including a forward primer, a Bst polymerase, and a monomer; and

FIG. 3 shows results of PAGE analysis of a product obtained by performing two extension reactions of a reaction mixture including template DNA fixed to a glass slide, a forward primer, a Bst polymerase, and a monomer.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects.

The terminology used herein is for the purpose of describing particular embodiments only and is not intended to be limiting. As used herein, “a,” “an,” “the,” and “at least one” do not denote a limitation of quantity, and are intended to cover both the singular and plural, unless the context clearly indicates otherwise. For example, “an element” has the same meaning as “at least one element,” unless the context clearly indicates otherwise. “Or” means “and/or.” “At least one” is not to be construed as limiting “a” or “an.” As used herein, the term “and/or” includes any and all combinations of one or more of the associated listed items. As used herein, the term “or” and “and/or” includes any and all combinations of one or more of the associated listed items. Expressions such as “at least one of,” when preceding a list of elements, modify the entire list of elements and do not modify the individual elements of the list.

It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within +20%, +10% or +5% of the stated value.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

According to an aspect of the disclosure, a method of producing a data-encoded nucleic acid, the method including performing one or more repeating cycles, each cycle comprising (i) an extension reaction and (ii) a removing monomer process, wherein the extension reaction is a polymerase extension of a data-encoded template DNA, the extension performed by incubating a reaction mixture comprising a buffer, the polymerase, the template, a primer having a region complementary to the template, and a first nucleotide monomer, such that the primer is extended in a template-dependent manner, wherein the removing monomer process (ii) is performed by removing unreacted first nucleotide monomer from the incubated reaction mixture in (i) to make a removed reaction mixture, or a second reaction mixture, and mixing the second or removed reaction mixture from which the unreacted nucleotide monomer is removed with a new nucleotide monomer wherein the incubating, removing, and mixing are repeated as one repeating cycle, wherein the template includes a coding region with a sequence complementary to the data-encoded nucleic acid and a primer-binding region complementary to a sequence of the primer, wherein the data-encoded nucleic acid includes modified nucleotides, and wherein the reaction mixture includes a modified nucleotide after performing one or more repetitions of the repeating cycle.

In one aspect, the method may further include incubating the reaction mixture including the template and the primer before performing the repeating cycle to obtain a product of hybridization of the template with the primer. The incubating may vary according to the selected template. The incubating may be performed under conditions suitable for denaturing and annealing double-stranded nucleic acids to obtain single strands of the primer and the template and hybridizing the template with the primer. The incubating may be performed at a high temperature, e.g., at a temperature of about 50° C. to about 100° C., about 80° C. to about 100° C., about 85° C. to about 95° C., about 80° C. to about 95° C., or about 87.5° C. to about 100° C. In addition, the incubating may be performed in an alkaline solution such as a NaOH aqueous solution.

In another aspect, the incubating may be performed isothermally in an optimal temperature range of the polymerase as known to a person of skill in the art The temperature may be, for example, in a range of ±5° C., ±2.5° C. or ±1.0° C. of the optimum temperature. The temperature may be, for example, a temperature suitable for obtaining activity of ±10%, ±5.0%, ±2.5%, or ±1.0% of optimal activity. An optimal temperature of a Bst polymerase may be about 65° C.

In one aspect, one terminal of the template may be fixed to a surface, e.g. a surface of a solid or a magnetic bead. In an aspect, the template may be biotinylated at the 5′-terminal and bound to a streptavidin-coated magnetic bead. By the fixation, a compplex of, or a product of, or a combination of, the primer, the template, and the polymerase may be selectively separated from other reaction products.

The incubating may refer to a process of repeatedly extending one nucleotide at a time in the reaction mixture not including a reversible terminator (RT). Because the reaction mixture does not use a reversible terminator, synthesis costs may be reduced, and since a deprotection process is not required, there is reduction in synthesis time. By ‘reversible terminator’ is meant a modified nucleotide that can terminate primer extension reversibly. Reversible terminators are known in the art, and may include a 3′-O-blocked reversible terminator and a 3′-O-unblocked reversible terminator.

In another aspect, the incubating may refer to extending nucleotides in the reaction mixture including the reversible terminator (RT). The reversible terminator is usually a modified nucleotide that reversibly terminates the extension of the primer. The reversible terminator may include, for example, a 3′-O-blocked reversible terminator and a 3′-O-unblocked reversible terminator.

In some aspects, the incubating may be performed under conditions sufficient for the polymerase to extend the primer in a template-dependent manner. The incubating may be performed under conditions capable of obtaining optimal polymerization activity of the polymerase. The incubating may be performed under conditions of a temperature, an ion concentration, or a buffer capable for obtaining optimal polymerization activity. The pH may include a pH range to obtain optimal activity of the polymerase. The buffer may be phosphate buffered saline (PBS). The ion may include Mg²⁺.

Conditions for the desired performance of a selected polymerase are well known in the art.

In some aspects, the polymerase may be a template-dependent polymerase. The polymerase may be a DNA or RNA polymerase or a reverse transcriptase, or a combination thereof. The polymerase may be selected from phi29 polymerase, Klenow fragment, Bst DNA polymerase, large fragment, T5 DNA polymerase, and M-MULV reverse transcriptase, or a combination thereof. The polymerase may be a natural or genetically-engineered polymerase, or a combination thereof.

In some aspects, the template may be a linear or circular nucleic acid. The template may be a single-stranded nucleic acid. The template may not include nucleotides that do not form Watson-Crick hydrogen bonds. The template may be DNA or RNA.

In some aspects, the modified nucleotide may be one forming Watson-Crick hydrogen bonds. The modified nucleotide may be located in a site not involved in Watson-Crick hydrogen bonds. The site may be located at a nucleic acid base. The modification may include a functional group at a nucleic acid base. The modified nucleotide may be a natural or non-natural nucleotide, or a combination thereof.

The modified nucleotide may be selected from 2-amino-2′-deoxyadenosine-5′-triphosphate, 5-bromo-2′-deoxycytidine-5′-triphosphate, 5-bromo-2′-deoxyuridine-5′-triphosphate, 7-deaza-2′-deoxyadenosine-5′-triphosphate, 7-deaza-2′-deoxyguanosine-5′-triphosphate, 2′-deoxyinosine-5′-triphosphate, 5-propynyl-2′-deoxycytidine-5′-triphosphate, 5-propynyl-2′-deoxyuridine-5′-triphosphate, 2′-deoxyuridine-5′-triphosphate, 5-fluoro-2′-deoxyuridine-5′-triphosphate, 5-iodo-2′-deoxycytidine-5′-triphosphate, 5-iodo-2′-deoxyuridine-5′-triphosphate, 5-methyl-2′-deoxycytidine-5′-triphosphate, 2-thiothymidine-5′-triphosphate, 2-thio-2′-deoxycytidine-5′-triphosphate, 5-aminoallyl-2′-deoxycytidine-5′-triphosphate, 5-aminoallyl-2′-deoxyuridine-5′-triphosphate, N4-methyl-2′-deoxycytidine-5′-triphosphate, 7-deaza-7-propargylamino-2′-deoxyadenosine-5′-triphosphate, 7-deaza-7-propargylamino-2′-deoxyguanosine-5′-triphosphate, 2′-deoxyadenosine-5′-triphosphate, 2′-deoxycytidine-5′-triphosphate, 2′-deoxyguanosine-5′-triphosphate, 2′-deoxythymidine-5′-triphosphate, biotin-16-aminoallyl-2′-dUTP, biotin-16-aminoallyl-2′-dCTP, desthiobiotin-6-aminoallyl-2′-deoxycytidine-5′-triphosphate, 2′-deoxyadenosine-5′-O-(1-thiotriphosphate), 2′-deoxycytidine-5′-O-(1-thiotriphosphate), 2′-deoxyguanosine-5′-O-(1-thiotriphosphate), 2′-deoxythymidine-5′-O-(1-thiotriphosphate), 5-aminoallylcytidine-5′-triphosphate, 2-aminoadenosine-5′-triphosphate 5-bromouridine-5′-triphosphate, 5-carboxycytidine-5′-triphosphate, 5-carboxymethylesteruridine-5′-triphosphate, 7-deazaadenosine-5′-triphosphate, 5-formylcytidine-5′-triphosphate, 5-formyluridine-5′-triphosphate, 5-hydroxycytidine-5′-triphosphate, 5-hydroxyuridine-5′-triphosphate, 5-hydroxymethylcytidine-5′-triphosphate, 5-hydroxymethyluridine-5′-triphosphate, 5-iodouridine-5′-triphosphate, 5-methoxycytidine-5′-triphosphate, 5-methoxyuridine-5′-triphosphate, N6-methyl-2-aminoadenosine-5′-triphosphate, 5-bromo-2′-deoxycytidine-5′-triphosphate, 5-propynyl-2′-deoxycytidine-5′-triphosphate, 5-propargylamino-2′-deoxycytidine-5′-triphosphate, cyanine 3-5-propargylamino-2′-deoxycytidine-5′-triphosphate, N4-biotin-OBEA-2′-deoxycytidine-5′-triphosphate, and biotin-16-aminoallyl-2′-dCTP. The modified nucleotide may be 5-methyl-dCTP, 5-hydroxy-dCTP, 5-carboxy-dCTP, or N6-methyl-dATP. In each repeating cycle, the reaction mixture may include a nucleotide monomer selected from dCTP, dATP, 5-methyl-dCTP, and N6-methyl-dATP, or a combination thereof.

In some aspects, the coding region of the template may be a sequence in which the same nucleotides are not consecutively located or a sequence in which two or less of the same nucleotide are not consecutively located. The coding region may be a sequence including T and G alternately arranged.

In other aspects, in each repeating cycle, the reaction mixture may include only one type of nucleotide monomer in the incubating process. The nucleotide monomer may be C or a modified nucleotide thereof, or A or a modified nucleotide thereof.

In some aspects, the encoded data may be a binary or n-nary code. The data encoded may be performed in the presence or absence of a certain nucleotide. The encoded data may be in any arbitrary form such as digital data or a nucleotide sequence. The encoded data may be in the form of a single nucleotide or a nucleotide sequence. The encoded data may include at least 1, 2, 3, 4, 5, 10, 15, 30, 50, 100, or 200 modified nucleotides. The encoded data may be encoded by each of the modified nucleotides or a combination of the modified nucleotides.

In some aspects, the removing the unreacted monomer may be performed under conditions not destroying a complex of, or a product of, or a combination of, the template, the primer, and the polymerase. In an aspect, conditions for removing the unreacted monomer do not affect the complex of, the combination of, or the product of, the template, the primer, and the polymerase.

In the method, removing of unreacted monomer may include washing the reaction mixture. Washing may be performed using water, an aqueous solution, or a buffer. Washing may be performed using at least one of any of, or a combination of, the buffers used for DNA polymerization, for example, a Bind and Wash (B&W) buffer (10 mM Tris-HCl pH 7.5, 1 mM EDTA, and 2.0 M NaCl), an isothermal amplification buffer (20 mM Tris-HCl, 10 mM (NH₄)₂SO₄, 50 mM KCl, 2 mM MgSO₄, 0.1% Tween® 20, and pH 8.8@25° C.), or a SSC buffer (150 mM sodium chloride, 15 mM sodium citrate, and pH 7.0).

In some aspects, the repeating cycle of the present method may be performed in a multiple parallel way, in a multiple reaction space. The repeating cycle may be performed by using a synthesis method selected from inkjet printing, electrochemical synthesis, and light-directed synthesis.

In some aspects, the method for producing a data-encoded nucleic acid may further include sequencing the data-encoded nucleic acid.

In other aspects, the method may further include preparing a template complementary to a sequence of the data-encoded nucleic acid. The preparing may be synthesizing. The synthesizing may be biological or chemical synthesizing.

In some aspects, in each repeating cycle, the nucleotide monomer may be added in the order according to the sequence of the data-encoded nucleic acid.

In some aspects, the encoded data may include an addressability sequence. The addressability sequence may include location information of data in the coding region.

In other aspects, the method for producing a data-encoded nucleic acid may further include: obtaining a nucleotide sequence of the data-encoded nucleic acid by sequencing the data-encoded nucleic acid; and decoding the encoded data from the nucleotide sequence of the data-encoded nucleic acid according to an encoding scheme. The sequencing may be performed by known methods, and may include MALDI-TOF and nanopore sequencing.

In some aspects, the encoding scheme may be bit information indicating the presence or absence of a certain nucleotide. The encoding scheme may be represented in the form of an encoding table.

According to another aspect of the disclosure, a data-encoded nucleic acid produced by the method may be provided.

According to the method of producing a data-encoded nucleic acid according to the aspect, the data-encoded nucleic acid may be produced efficiently.

According to another aspect of the disclosure, the data-encoded nucleic acid may be used for encoding and decoding data.

### EXAMPLES

Hereinafter, the disclosure will be described in more detail with reference to the following examples. However, the following examples are merely presented to exemplify the disclosure, and the scope of the disclosure is not limited thereto.

### Example 1: Template-Dependent Synthesis of Data-Encoded DNA in Liquid

In this example, data-encoded DNA was synthesized in a template-dependent manner by incubating in a solution including a template DNA, a primer, and a DNA polymerase.

A template DNA with a length of 50 bp was designed. The template DNA was a single-stranded DNA having a nucleotide sequence of SEQ ID NO: 1. The template DNA included, in a 5′ to 3′ direction, a reverse primer-binding region, a data coding region, and a forward primer-binding region. The reverse primer-binding region, the data coding region, and the forward primer-binding region had lengths of 10 bp, 20 bp, and 20 bp, corresponding to positions 1 to 10, 11 to 30, and 31 to 50 of SEQ ID NO: 1, respectively. The reverse primer and the forward primer were designed to identify a synthesized, extended primer by PCR amplification. The forward primer provides a starting 3′-OH for the template-dependent synthesis of the nucleic acid.

The template DNA bound to biotin at the 5′-end was purchased from an external company. Biotin bound to the 5′-end of the template DNA was used to bind to streptavidin magnetic beads after each cycle of the DNA synthesis reaction in a solution. The forward primer has a nucleotide sequence of SEQ ID NO: 2. The forward primer was designed to bind by complementary binding under optimal temperature conditions for the polymerase being used. In this example, the forward primer was designed to bind at about 65° C. which is the optimal temperature for Bst polymerase. The data coding region is a sequence in which T and G are alternately repeated. Accordingly, only one nucleotide is used for extension in one cycle of the reaction while the DNA polymerase synthesizes DNA in a template-dependent manner in the DNA synthesis or extension reaction. In this case, each reaction mixture includes one nucleotide monomer as the nucleotide complementary to the data coding region of the template DNA. The monomer can be selected from any natural nucleotide or any modified nucleotide as long as it is complementary to the coding region of the template DNA. Therefore, for each nucleotide of the data coding region, the number of nucleotides that can be linked to the end of the extending primer is equal to the number of nucleotides that can form a complementary bond with said each nucleotide of the coding region. This indicates that if there are n-nucleotides that can form a complementary bond with each nucleotide of the coding region, the end of the extending primer can store n-nary information. In addition, the natural nucleotide and the modified nucleotide may be those recognized by the polymerase and capable of catalyzing ligation.

The DNA synthesis reaction may repeatedly extend one nucleotide at a time in the reaction mixture not including a reversible terminator (RT). Because the DNA synthesis reaction does not use the reversible terminator, synthesis costs may be reduced, and since a deprotection process is not required, synthesis time is reduced. The reversible terminator refers to a modified nucleotide that reversibly terminates the extension of the primer. The reversible terminator includes a 3′-O-blocked reversible terminator and a 3′-O-unblocked reversible terminator.

In more detail, 2 μM of the biotinylated template DNA of SEQ ID NO: 1, an isothermal amplification buffer (20 mM Tris-HCl, 10 mM (NH₄)₂SO₄, 50 mM KCl, 2 mM MgSO₄, 0.1% Tween® 20, and pH 8.8@25° C.), 1 U of Bst DNA polymerase, 4 mM of MgSO₄, 100 UM of a monomer (one of dATP, dCTP, d6mATP, and d5mCTP), and 10 UM of a forward primer of SEQ ID NO: 2 were mixed in a nuclease-free, water-containing polypropylene tube to prepare a reaction mixture. Table 1 below shows, volumes concentrations of each reagent used in the example composition.

TABLE 1

| Composition | Volume (50 μL) | | Concentration | |
|---|---|---|---|---|
| 20 μM template DNA | 5 | μL | 2 μM (0.1 nmol) | |
| 100 μM F-Primer | 5 | μL | 10 | μM |
| 1 mM dNTP | 5 | μL | 100 | μM |
| Bst 2.0 polymerase (0.33 | 3 | μL | 1 U (0.02 U/μL) | |
| U/μL) | | | | |
| 10X Isothermal amplification | 5 | μL | 1X | |
| buffer | | | | |
| 100 mM MgSO4 | 2 | μL | 4 | mM |

| | | | |
|---|---|---|---|
| Magnetic beads (10 mg/mL) | 20 | μL | Beads 0.2 mg (ssDNA |
| | | | 0.1 nmol) |
| Distilled water | 5 | μL | — |

The d6mATP and d5mCTP represent M6-methyl-dATP and 5-methyl-dCTP, respectively. In this regard, C and d5mCTP, or A and domATP may represent binary codes 0 and 1, respectively. After the reaction mixture-containing tube was incubated at 95° C. for 3 minutes, the tube was cooled, and then the tube was incubated at 70° C. for 30 seconds and cooled to 4° C. This process induces hybridization of the forward primer and the template DNA.

0.2 mg of streptavidin-coated magnetic beads were added to the tube and incubated at room temperature for 15 minutes to induce binding of the 5′-biotin of the template DNA to the streptavidin coated magnetic beads. Addition of the magnetic beads to the reaction mixture may be performed at any stage of the reaction as long as biotin binds to streptavidin and the bond is not broken.

The reaction mixture was incubated at 65° C. for 1 hour to induce template DNA-dependent extension from the primer. Hereinafter, this process is referred to as DNA extension process. The extension reaction may be performed in an optimal temperature range for the chosen DNA polymerase. The temperature may be, for example, in a range of +5° C., +2.5° C., or +1.0° C. of the optimal temperature. The temperature may be, for example, a temperature to obtain activity of +10%, +5.0%, +2.5%, or +1.0% of optimal activity.

Upon completion of the extension reaction, the tube was placed under a magnetic field to fix the magnetic beads which were bound to the complex of the template DNA, the primer extended in a template-dependent manner, and DNA polymerase, and the reaction mixture contained in the tube was washed three times with a Bind and Wash (B&W) buffer (10 mM Tris-HCl PH 7.5, 1 mM EDTA and 2.0 M NaCl) in order to remove the monomer. Hereinafter, this process is referred to as washing process. The washing process may be to ensure that the primer, the template, and the DNA polymerase are not removed from the reaction product and can be used in the extension reaction in the next extension step.

However, the washing step is merely an example of the step of removing the monomer, and the step of removing the monomer should not be interpreted as necessarily including a washing step. As an example, the magnetic beads can be separated from the reaction solution containing the monomer by simple solid-liquid separation. Therefore, the next extension reaction can be performed on the “complex containing the template DNA, the extended primer, and DNA polymerase” separated by simple solid-liquid separation without the “washing step”.

The DNA extension process and the washing process were repeated to synthesize DNA. In this regard, a new monomer was added to the reaction mixture of every cycle. Upon completion of all extension reactions, the tube was incubated at 90° C. for 3 minutes to separate the extended primer from the template DNA by denaturation. Subsequently, the magnetic beads bound to the template DNA were fixed using a magnet, and then the liquid containing the separated, extended primer was recovered from the reaction mixture. The removal of the extended primer from the complex of the extended primer and the template DNA may be performed not only by the heat treatment but also by using a denaturing agent, e.g., an alkaline solution such as NaOH.

The extended primer contained in the recovered liquid was quantified. The quantification was performed by Qubit fluorometric quantification or electrophoresis such as polyacrylamide gel electrophoresis (PAGE). In addition, the extended primer contained in the recovered liquid was sequenced. The sequence may be analyzed by any known method, for example, MALDI-TOF, nanopore sequencing, and electrophoresis such as PAGE. The quantification and sequencing of the extended primer may also be performed by any known method such as PCR.

The encoded information may be decoded from the extended primer by the any sequencing method. The decoding may be performed according to a pre-set coding table. In the coding table, a number may be assigned according to a type of base introduced into each position of the extended primer. For example, in the case where 15 modified dCTPs in addition to dCTP, i.e., a total of 16 dCTPs, may be introduced into one position of the extended primer, information with 4 bits may be encoded. Accordingly, the use of modified bases may increase bit density in a nucleotide. The dCTP and 15 modified dCTPs may be dCTP, 5-methyl-dCTP, 5-hydroxy-dCTP, 5-hydroxymethyl-dCTP, N⁴-methyl-dCTP, 5-aminoallyl-dCTP, 5-formyl-dCTP, 5-carboxy-dCTP, 5-iodo-dCTP, 5-Br-dCTP, 5-propynyl-dCTP, 5-propargylamino-dCTP, cyanine 3-dCTP, desthiobiotin-6-dCTP, N⁴-biotin-dCTP, and biotin-16-dCTP.

Nucleotide modification may include any modification as long as the modification is recognized by the polymerase and used for the extension. The modification may be located at a site not involved in complementary hydrogen bonds in a double-stranded DNA. The modification may be a functional group added to the base of the nucleic acid.

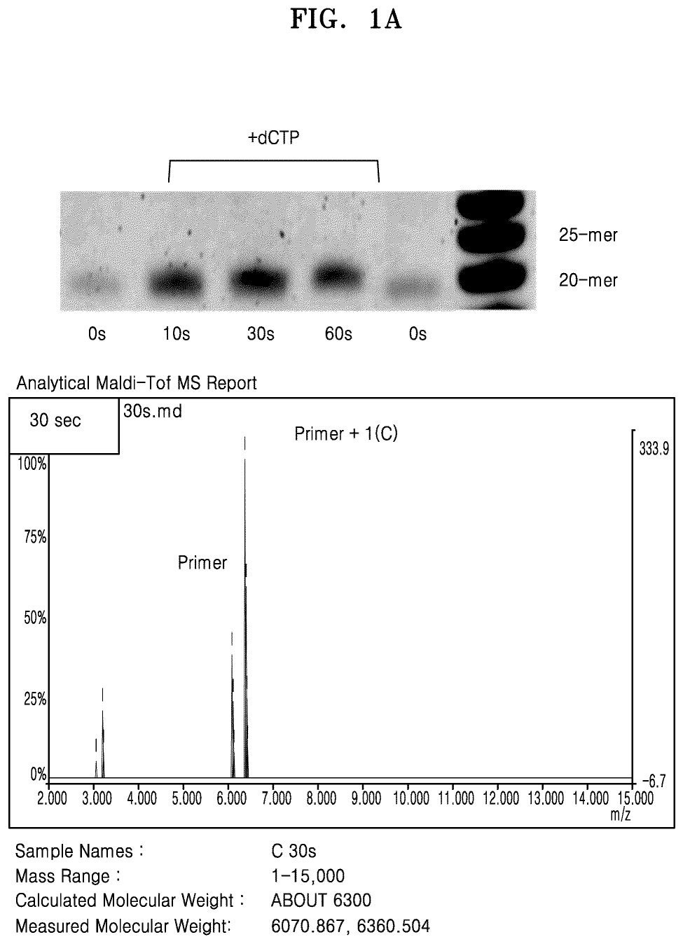

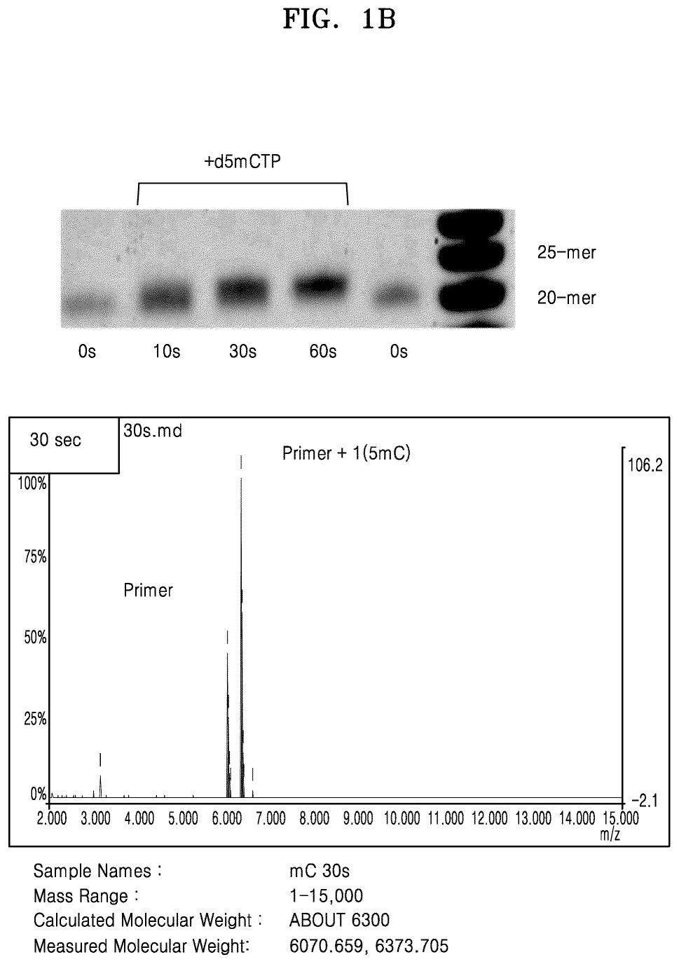

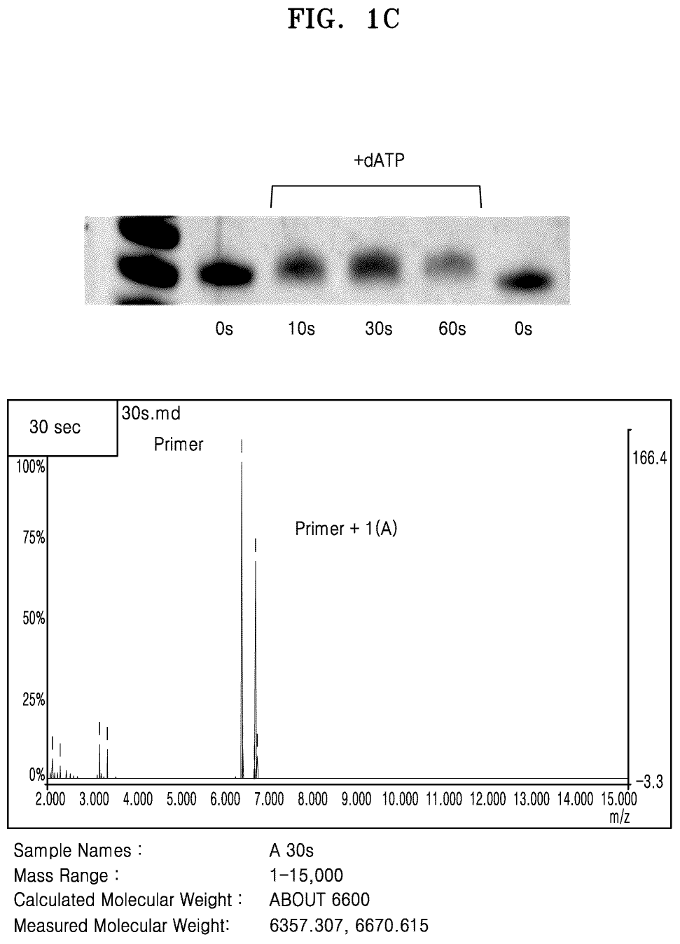

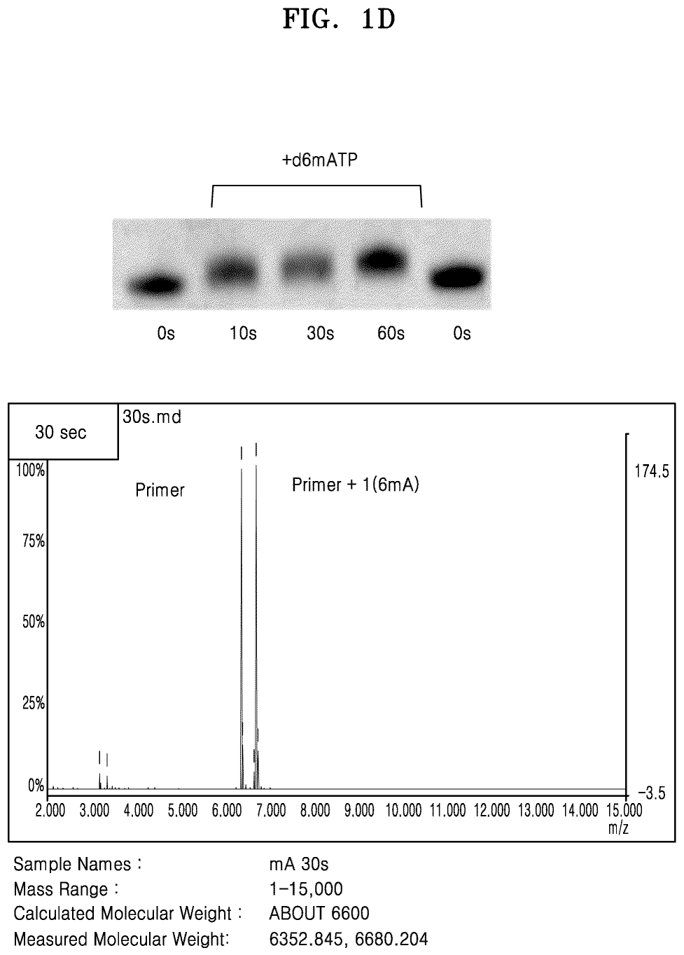

FIGS. 1A and 1B show results of PAGE (upper) analysis and MALDI-TOF (lower) analysis of a product obtained by performing one extension reaction of a reaction mixture including a forward primer, a Bst polymerase, and a monomer. The forward primer was a primer of SEQ ID NO: 2 having a length of 20 bp and a molecular weight of about 6067 Da, and the monomers were dCTP (C) and 5-methyl-dCTP (5mC), respectively. As shown in the upper sides of FIGS. 1A and 1B, reaction products were identified at 10 seconds, 30 seconds, and 60 seconds after initiation of the reaction. As shown in the lower sides of FIGS. 1A and 1B, as a result of analyzing the samples obtained after 30 seconds from the initiation of reaction of the upper sides of FIGS. 1A and 1B by MALDI-TOF, the primer and the primer linked to one nucleotide, i.e., C or 5mC, were identified at expected molecular weight positions.

FIGS. 1C and 1D show results of PAGE-gel analysis and MALDI-TOF analysis of a product obtained by further performing one extension reaction of the reaction mixture including reaction products obtained by washing the resultant of FIG. 1A or 1B, and adding Bst polymerase, and a monomer. The monomers added here were dATP (A) and N6-methyl-dATP (6 mA), respectively. This indicates that although the reaction product obtained in the previous extension process was washed, the extended primer, the template, and the polymerase were not removed, and were available for the next extension process.

As shown in the upper sides of FIGS. 1C and 1D, reaction products were identified at 10 seconds, 30 seconds, and 60 seconds after initiation of the reaction. As shown in the lower sides of FIGS. 1C and 1D, as a result of analyzing the samples obtained after 30 seconds from the initiation of reaction of the upper sides of FIGS. 1C and 1D by MALDI-TOF, the primer and the primer linked to one nucleotide, i.e., A or 5 mA, were identified at expected molecular weight positions.

This indicates that the Bst polymerase may catalyze the extension reaction by using not only natural C and A but also modified 5mC and 6 mA as substrates. The extension reaction may be performed in parallel in a reactor including a plurality of tubes or wells containing separate monomers. Such parallel extension reaction allows synthesis of a nucleic acid storing desired information in large quantities within a short period of time. The parallel extension reaction may be performed by known methods. The parallel extension reaction may be performed on a spot of a microarray.

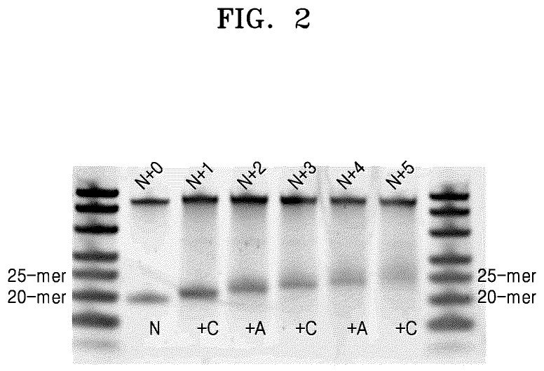

FIG. 2 shows results of PAGE analysis of a product obtained by performing five extension reactions with a reaction mixture including a forward primer, a Bst polymerase, and a monomer. The forward primer was a primer of SEQ ID NO: 2 with a length of 20 bp and a molecular weight of about 6067 Da, and the monomers used therein were dCTP (C), dATP (A), dCTP (C), dATP (A), and dCTP (C), respectively. As shown in FIG. 2, as a result of performing the reaction of extending by one nucleotide five times, products with lengths of 20 bp, 21 bp, 22 bp, 23 bp, 24 bp, and 25 bp were confirmed. In FIG. 2, N indicates a primer with a length of 20 bp used as a control.

This indicates that although the reaction product obtained in the previous extension process was repeatedly washed five times, the extended primer, the template, and the polymerase were not removed and were available to perform additional extension reactions. The washing was performed at room temperature using an isothermal reaction buffer.

### Example 2: Template-Dependent Synthesis of Data-Encoded DNA on Surface of Solid

In this example, data-encoded DNA was synthesized in a template-dependent manner by incubating a reaction mixture including a primer, a polymerase and a template DNA fixed to a surface of a solid surface, a glass slide with a size of 25 mm×75 mm×1 mm. The template DNA and the DNA primer used had SEQ ID NOS: 1 and 2, respectively. The 5′-end of the template DNA was modified with an amine group (NH₂) and reacted with a N-hydroxycuccinimide (NHS)-coated glass slide. Bst polymerase was used.

The NHS-coated glass slide is a commercially available product, CodeLink® Activated Slides. The glass slide was designed to be coated with a hydrophilic polymer including a NHS ester reactive group and minimize non-specific binding.

A template DNA of SEQ ID NO: 1 having the 5′-end modified with an amine group (NH₂) was purchased from an external company. The NHS-coated glass slide was fixed to a reaction compartment of a thermocycler equipped with a Peltier element so that the temperature of the reaction mixture may be controlled. Ten μM of the amine-modified template DNA was added to a sodium phosphate buffer (50 mM, pH 8.5) in the reaction compartment, and the reaction mixture was incubated at room temperature for 12 hours without stirring to bind the template DNA to the surface of the slide. A volume of 200 mL of a 50 mM ethanolamine-containing aqueous solution (Tris 0.1M, pH 9) was added to the reacted glass slide and incubated at 50° C. for 30 minutes without stirring to block unreacted NHS groups. Upon completion of the reaction, the glass slide was washed with distilled water. Polymerization was performed according to the following processes.

### Primer Hybridization Process

A 300 μL volume of a primer hybridization solution (a 1 μM forward primer, SSC buffer (150 mM sodium chloride, 15 mM sodium citrate, and pH 7.0) 4×, 0.1% SDS, and distilled water) was added to the reaction compartment having the glass slide to which the template DNA of SEQ ID NO: 1 was fixed. The compartment was sealed followed by incubation at 40° C. for 2 hours and washing with a 4×SSC buffer. The reaction compartment was then filled with a 2×SSC buffer (0.1% SDS), and incubation of the reaction mixture at 40° C. for 10 minutes was performed twice. The glass slide was then washed with 50 mL of a 0.2×SSC buffer at 25° C. for 1 minute and then washed with 50 mL of a 0.1×SSC buffer at 25° C. for 1 minute.

As the forward primer, a primer having a nucleotide sequence of SEQ ID NO: 2 and labeled with Cy3, i.e., tetramethylrhodamine (TRITC), was used. Cy3 is a fluorescent dye with a light orange color and has a peak excitation wavelength and an emission wavelength of at 554 nm and 568 nm, respectively. Cy3 dye was covalently linked to the 5′-terminal phosphate group of the primer activated by NHS ester via a covalent bond, Fixation of the template DNA to the glass slide was confirmed by visualizing the fluorescent primer hybridized with the template DNA.

### Bst 2.0 Polymerase Binding Process

150 μL of an isothermal amplification buffer containing 1U of the Bst 2.0 polymerase was added to the reaction compartment, followed by incubation at 25° C. for 30 minutes.

dCTP Extension Process

150 μL of an isothermal amplification buffer containing 200 UM of dCTP was added to the reaction compartment, followed by incubation at 65° C. for 1 minute. In this case, the monomer may be, for example, C and 5-methyl-dCTP (d5mCTP), and binary codes thereof may represent 0 and 1.

### Washing Process

The reaction compartment was washed three or four times with an isothermal amplification buffer.

dATP Extension Process

150 μL of an isothermal amplification buffer containing 200 UM of dATP was added to the reaction compartment, followed by incubation at 65° C. for 1 minute. In this case, the monomer may be, for example, A and M6-methyl-dATP (d6mATP), and binary codes thereof may represent 0 and 1.

### Final Washing Process

The glass slide was washed in 50 mL of the 0.1×SSC buffer for 1 minute.

### Denaturation Process

The reaction product was denatured using nuclease-free water at 95° C. for 3 minutes. The reaction product was analyzed by electrophoresis using denaturation PAGE to identify the length of the product DNA.

Once the hybridization process, the extension process, and the washing process are completed, a cycle of extending one nucleotide is completed. This cycle may be repeated by the number of extended nucleotides.

Upon completion of the desired number of extension reactions for a desired number of nucleotides, the reaction mixture was incubated at 95° C. for 3 minutes in the wash isothermal amplification buffer to separate the extended primer from the template DNA by denaturation. The extended primer was recovered and size and quantity thereof were analyzed by PAGE.

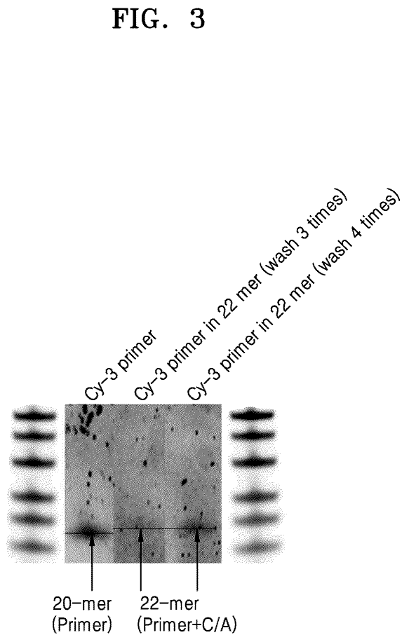

FIG. 3 shows results of PAGE analysis of a product obtained by performing two extension reactions on a reaction mixture including a template DNA fixed to a glass slide, a forward primer, a Bst polymerase, and a monomer. The forward primer was a primer of SEQ ID NO: 2 having a length of 20 bp and a molecular weight of about 6067 Da, and the monomers were dCTP (C) and dATP (A), sequentially. As shown in FIG. 3, an extended primer band was identified at 22 bp position that is increased from the 20 bp position of the control. That is, as the initial primer with 20 bp was extended with A and C and thus an extended primer with 22 bp was synthesized. Although washing was performed three times and four times, synthesis was not affected thereby.

In FIG. 3, in the control, the reaction mixture did not include any monomers other than the Cy3-labeled primer.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. A method of producing a data-encoded nucleic acid, the method comprising performing one or more repeating cycle of an extension reaction (i) followed by a removing monomer process (ii) wherein:
the extension reaction (i) is a polymerase extension of a data-encoded template DNA wherein the polymerase is incubated with a reaction mixture including a data-encoded template, a primer including a region complementary to the template, a buffer, and a first nucleotide monomer, to produce an incubated reaction mixture;
the removing monomer process (ii) removes unreacted first nucleotide monomer from the extension reaction (i) to produce a removed reaction mixture, and mixing a second monomer in the removed reaction mixture to repeat the extension reaction (i);
wherein the data-encoded template comprises:
a coding region with a sequence complementary to the data-encoded nucleic acid; and
a primer-binding region complementary to a sequence of the primer,
wherein the data-encoded nucleic acid includes a modified nucleotide, and
wherein the reaction mixture obtained after one or more repetitions of the repeating cycle includes the modified nucleotide.

2. The method of claim 1, further comprising incubating the reaction mixture including the template and the primer to obtain a product of hybridization of the template with the primer before performing the repeating cycle.

3. The method of claim 1, wherein the incubating is performed under conditions sufficient for the polymerase to extend the primer in a template-dependent manner.

4. The method of claim 1, wherein the incubating is performed isothermally in an optimal temperature range of the polymerase.

5. The method of claim 1, wherein the template does not comprise a nucleotide not forming Watson-Crick hydrogen bonds.

6. The method of claim 1, wherein the modified nucleotide is a natural or non-natural nucleotide.

7. The method of claim 1, wherein the modified nucleotide is selected from 2-amino-2′-deoxyadenosine-5′-triphosphate, 5-bromo-2′-deoxycytidine-5′-triphosphate, 5-bromo-2′-deoxyuridine-5′-triphosphate, 7-deaza-2′-deoxyadenosine-5′-triphosphate, 7-deaza-2′-deoxyguanosine-5′-triphosphate, 2′-deoxyinosine-5′-triphosphate, 5-propynyl-2′-deoxycytidine-5′-triphosphate, 5-propynyl-2′-deoxyuridine-5′-triphosphate, 2′-deoxyuridine-5′-triphosphate, 5-fluoro-2′-deoxyuridine-5′-triphosphate, 5-iodo-2′-deoxycytidine-5′-triphosphate, 5-iodo-2′-deoxyuridine-5′-triphosphate, 5-methyl-2′-deoxycytidine-5′-triphosphate, 2-thiothymidine-5′-triphosphate, 2-thio-2′-deoxycytidine-5′-triphosphate, 5-aminoallyl-2′-deoxycytidine-5′-triphosphate, 5-aminoallyl-2′-deoxyuridine-5′-triphosphate, N4-methyl-2′-deoxycytidine-5′-triphosphate, 7-deaza-7-propargylamino-2′-deoxyadenosine-5′-triphosphate, 7-deaza-7-propargylamino-2′-deoxyguanosine-5′-triphosphate, 2′-deoxyadenosine-5′-triphosphate, 2′-deoxycytidine-5′-triphosphate, 2′-deoxyguanosine-5′-triphosphate, 2′-deoxythymidine-5′-triphosphate, biotin-16-aminoallyl-2′-dUTP, biotin-16-aminoallyl-2′-dCTP, desthiobiotin-6-aminoallyl-2′-deoxycytidine-5′-triphosphate, 2′-deoxyadenosine-5′-O-(1-thiotriphosphate), 2′-deoxycytidine-5′-O-(1-thiotriphosphate), 2′-deoxyguanosine-5′-O-(1-thiotriphosphate), 2′-deoxythymidine-5′-O-(1-thiotriphosphate), 5-aminoallylcytidine-5′-triphosphate, 2-aminoadenosine-5′-triphosphate 5-bromouridine-5′-triphosphate, 5-carboxycytidine-5′-triphosphate, 5-carboxymethylesteruridine-5′-triphosphate, 7-deazaadenosine-5′-triphosphate, 5-formylcytidine-5′-triphosphate, 5-formyluridine-5′-triphosphate, 5-hydroxycytidine-5′-triphosphate, 5-hydroxyuridine-5′-triphosphate, 5-hydroxymethylcytidine-5′-triphosphate, 5-hydroxymethyluridine-5′-triphosphate, 5-iodouridine-5′-triphosphate, 5-methoxycytidine-5′-triphosphate, 5-methoxyuridine-5′-triphosphate, N6-methyl-2-aminoadenosine-5′-triphosphate, 5-bromo-2′-deoxycytidine-5′-triphosphate, 5-propynyl-2′-deoxycytidine-5′-triphosphate, 5-propargylamino-2′-deoxycytidine-5′-triphosphate, cyanine 3-5-propargylamino-2′-deoxycytidine-5′-triphosphate, N4-biotin-OBEA-2′-deoxycytidine-5′-triphosphate, and biotin-16-aminoallyl-2′-dCTP.

8. The method of claim 1, wherein, in each repeating cycle, the reaction mixture comprises a nucleotide monomer selected from dCTP, dATP, dGTP, dTTP, 2-amino-2′-deoxyadenosine-5′-triphosphate, 5-bromo-2′-deoxycytidine-5′-triphosphate, 5-bromo-2′-deoxyuridine-5′-triphosphate, 7-deaza-2′-deoxyadenosine-5′-triphosphate, 7-deaza-2′-deoxyguanosine-5′-triphosphate, 2′-deoxyinosine-5′-triphosphate, 5-propynyl-2′-deoxycytidine-5′-triphosphate, 5-propynyl-2′-deoxyuridine-5′-triphosphate, 2′-deoxyuridine-5′-triphosphate, 5-fluoro-2′-deoxyuridine-5′-triphosphate, 5-iodo-2′-deoxycytidine-5′-triphosphate, 5-iodo-2′-deoxyuridine-5′-triphosphate, 5-methyl-2′-deoxycytidine-5′-triphosphate, 2-thiothymidine-5′-triphosphate, 2-thio-2′-deoxycytidine-5′-triphosphate, 5-aminoallyl-2′-deoxycytidine-5′-triphosphate, 5-aminoallyl-2′-deoxyuridine-5′-triphosphate, N4-methyl-2′-deoxycytidine-5′-triphosphate, 7-deaza-7-propargylamino-2′-deoxyadenosine-5′-triphosphate, 7-deaza-7-propargylamino-2′-deoxyguanosine-5′-triphosphate, 2′-deoxyadenosine-5′-triphosphate, 2′-deoxycytidine-5′-triphosphate, 2′-deoxyguanosine-5′-triphosphate, 2′-deoxythymidine-5′-triphosphate, biotin-16-aminoallyl-2′-dUTP, biotin-16-aminoallyl-2′-dCTP, desthiobiotin-6-aminoallyl-2′-deoxycytidine-5′-triphosphate, 2′-deoxyadenosine-5′-O-(1-thiotriphosphate), 2′-deoxycytidine-5′-O-(1-thiotriphosphate), 2′-deoxyguanosine-5′-O-(1-thiotriphosphate), 2′-deoxythymidine-5′-O-(1-thiotriphosphate), 5-aminoallylcytidine-5′-triphosphate, 2-aminoadenosine-5′-triphosphate 5-bromouridine-5′-triphosphate, 5-carboxycytidine-5′-triphosphate, 5-carboxymethylesteruridine-5′-triphosphate, 7-deazaadenosine-5′-triphosphate, 5-formylcytidine-5′-triphosphate, 5-formyluridine-5′-triphosphate, 5-hydroxycytidine-5′-triphosphate, 5-hydroxyuridine-5′-triphosphate, 5-hydroxymethylcytidine-5′-triphosphate, 5-hydroxymethyluridine-5′-triphosphate, 5-iodouridine-5′-triphosphate, 5-methoxycytidine-5′-triphosphate, 5-methoxyuridine-5′-triphosphate, N6-methyl-2-aminoadenosine-5′-triphosphate, 5-bromo-2′-deoxycytidine-5′-triphosphate, 5-propynyl-2′-deoxycytidine-5′-triphosphate, 5-propargylamino-2′-deoxycytidine-5′-triphosphate, cyanine 3-5-propargylamino-2′-deoxycytidine-5′-triphosphate, N4-biotin-OBEA-2′-deoxycytidine-5′-triphosphate, and biotin-16-aminoallyl-2′-dCTP.

9. The method of claim 1, wherein a terminal of the template is fixed to a surface of a solid.

10. The method of claim 1, wherein, in each repeating cycle, the reaction mixture comprises only one type of nucleotide monomer.

11. The method of claim 1, wherein the polymerase is selected from phi29 polymerase, Klenow fragment, Bst DNA polymerase, large fragment, Bsu DNA polymerase, large fragment, T5 DNA polymerase, and M-MULV reverse transcriptase.

12. The method of claim 1, wherein the removing of the unreacted monomer comprises washing the reaction mixture.

13. The method of claim 1, wherein in the removing process, complexes of the template, the primer, and the polymerase are not removed from the reaction mixture.

14. The method of claim 1, further comprising designing a sequence of the data-encoded nucleic acid.

15. The method of claim 1, further comprising preparing a template complementary to a sequence of the data-encoded nucleic acid.

16. The method of claim 1, wherein the nucleotide monomer is sequentially added in accordance with a sequence of the data-encoded nucleic acid in each repeating cycle.

17. The method of claim 1, further comprising:
obtaining a nucleotide sequence of the data-encoded nucleic acid by sequencing the data-encoded nucleic acid; and
decoding encoded data from the nucleotide sequence of the data-encoded nucleic acid according to an encoding scheme.

18. A data-encoded nucleic acid produced by the method of claim 1.


SEQUENCE LISTING (2 sequences)

SEQ ID NO: 1 - DNA, 50 bp, synthetic construct
  source 1..50: mol_type = other DNA
gacgtctgtatgtgtgtgtgtgtgtgtgtgagcttcgtgtagagccgagt

SEQ ID NO: 2 - DNA, 20 bp, synthetic construct
  source 1..20: mol_type = other DNA
actcggctctacacgaagct